(19) United States
(12) Patent Application Publication
(10) Pub. No.: US 2010/0228337 A1
(43) Pub. Date: Sep. 9, 2010

(54) MIRROR IMAGE STENT AND METHOD OF USE

(75) Inventor: OBRADOVIC MILISAV, Loerrach (DE)

Correspondence Address: FULWIDER PATTON, LLP (ABBOTT), 6060 CENTER DRIVE, 10TH FLOOR, LOS ANGELES, CA, 90045, US

(73) Assignee: ABBOTT LABORATORIES VASCULAR ENTERPRISES LIMITED, Dublin (DE)

(21) Appl. No.: 12/397,817

(22) Filed: Mar. 4, 2009

(51) Int. Cl. A61F 2/06

(52) U.S. Cl. 623/1.16

(57) ABSTRACT

that at least a proximal end ring in combination with its associated links is the mirror image of a distal end ring and its associated links. The rings that form the proximal end and the rings that form the distal end have a radial stiffness associated therewith, and said stiffness is greater than a stiffness of the rings that form the interior portion of the stent. The dual stiffness of the stent leads to a more uniform expansion in the presence of uneven radial forces on the stent during the expansion process.

### BACKGROUND

The invention relates to vascular repair devices, and in particular intravascular stents, which are adapted to be implanted into a patient's body lumen, such as a blood vessel or coronary artery, to maintain the patency thereof. Stents are particularly useful in the treatment of atherosclerotic stenosis in arteries and blood vessels.

Stents are generally tubular-shaped devices which function to hold open a segment of a blood vessel or other body lumen such as a coronary artery. They also are suitable for use to support and hold back a dissected arterial lining that can occlude the fluid passageway. One method and system developed for delivering stents to a desired location within the patient's body lumen involves crimping a stent about an expandable member (e.g., a balloon) on the distal end of a catheter, advancing the catheter through the patient's vascular system until the stent is in the desired location, and then inflating the expandable member to expand the stent within the blood vessel. The expandable member is then deflated and the catheter withdrawn, leaving the expanded stent inside the blood vessel to hold open the passageway. Advancing the stent through a patient's vasculature, which can involve traversing sharp bends and other obstacles, requires that the stent have a fair degree of flexibility. Stent flexibility also permits the stent to be deployed in and conform to a curved section of a patient's vasculature.

At present, there are numerous commercial stents being marketed throughout the world. While some of these stents are flexible and have the appropriate radial rigidity needed to hold open a vessel or artery, there typically is a tradeoff between flexibility, radial strength, and the ability to tightly compress or crimp the stent onto a catheter so that it does not move relative to the catheter or dislodge prematurely prior to actuation in a vessel. The ability to securely affix a stent onto the balloon so that it will not be inadvertently dislodged during the delivery of the stent is a primary concern.

A critical characteristic of the stent is the stiffness of the stent, which affects the capacity of the stent to be crimped onto the delivery modality, but also controls the expansion of the stent and the ultimate strength of the stent in withstanding radial forces to retain the body lumen in the expanded configuration. If the stiffness is too high, the stent will lack the flexibility to traverse the curves of the body lumen and will be difficult to position accurately. If the stiffness is too low, the stent will lack the rigidity to reliably carry out is function as a support for the body lumen. Thus, selecting a stiffness of a stent is a critical design choice, and one that is inherently fraught with diametrically opposed objects.

The stiffness of the stent also affects the deployment within the body lumen. When a stent is delivered to a body lumen, the underlying balloon may be inflated from its delivery state to its inflated state to expand the stent. It is vital that the stent expand evenly across the length of the stent so as not to expand one side or portion earlier than another. If the stent expands more quickly in one location, it may translate from its original position as the stent slides within the body lumen. Uneven expansion can also lead to tissue damage as the stent may over-expand in one location in order to be positioned correctly and expanded across the length of the stent.

Although a constant expansion is desired, one adverse condition that is common in stent deployment is referred to as a “dog bone” effect, where the stent expands initially more quickly at the ends and more slowly in the middle of the stent. The expansion at the ends of the stent occur because the balloon is typically longer than the stent to ensure that the entire length of the stent is expanded. The excess balloon area outside the stent expands with less resistance comparatively, which in turn applies a higher force on the ends of the stent. While the forces on the ends of the stent are higher, the forces on the middle of the stent are not affected by the excess balloon material and are therefore less than the ends. Because the stiffness of the stent is constant across the stent, the higher forces at the end of the stent results in the ends expanding earlier and faster than the middle portion, causing the dog bone effect. This uneven expansion of the stent at the respective ends can lead to problems in placing the stent at the desired location as stent drift may result. Further, the uneven expansion can lead to poor adhesion characteristics and result in dislodgement of the stent, as well as damage to the adjacent tissue.

In addition, as the stent is manipulated to traverse the circuitous path of the coronary artery or other desired locations within the patient's body lumens, bending forces are imposed on the stent. The forces tend to be highest at the end positions, whereas the bending forces tend to be more evenly distributed in the middle portion of the stent. Designing a stent that will be sufficiently stiff to withstand the higher bending forces at the ends of the stent leads to a stent that is less flexible than optimum.

What has been needed and heretofore unavailable is a stent which has a high degree of flexibility so that it can be advanced through tortuous passageways and can be readily expanded, and yet have the mechanical strength to hold open the body lumen or artery into which it is implanted and provide adequate vessel wall coverage. In particular, it would be desirable to have a stent that is designed to expand more evenly than prior art stents without experiencing a dog bone effect, and accommodate the experienced high bending forces at the ends of the stent without unduly limiting the flexibility of the stent. The present invention satisfies these and other needs. That is, the stent of the present invention has variable stiffness that includes a higher stiffness at the ends of the stent and a lower stiffness at the middle of the stent. The variable stiffness allows for improved stent retention and expansion characteristics while preserving the strength capabilities to address the high bending forces at the ends of the stent.

### SUMMARY OF THE INVENTION

The present invention is directed to a stent configuration that provides for a more balanced end-to-end radial force distribution and for more homogeneous stent expansion. Additionally, the configuration provides for enhanced stent retention on an expansion balloon prior to deployment.

The stent is comprised of a series of undulating rings that are linked to one another such that the rings each extend about a common axis and thereby define a cylindrical structure. The undulating rings are in turn each comprised of a series of undulations that are interconnected by struts. Such pattern allows the ring, and hence the stent, to expand about its central axis so as to increase the cylindrical structure's diameter. The undulations may be referred to as peaks and valleys wherein the peaks may be regarded as those undulations that point towards the distal end of the stent while valleys are regarded as the undulations that point toward the proximal end of the stent. Adjacent rings are either linked peak-to-peak, valley-to-valley or valley-to-peak. The stent has at least one plane of symmetry perpendicular to its longitudinal axis such that at least the proximal end ring in combination with its associated links is the mirror image of the distal end ring and its associated links. The rings that form the proximal end and the rings that form the distal end have a radial stiffness associated therewith, and said stiffness is greater than a stiffness of the rings that form the interior portion of the stent. The dual stiffness of the stent leads to a more uniform expansion in the presence of uneven radial forces on the stent during the expansion process.

These and other features and advantages of the present invention will become apparent from the following detailed description of the preferred embodiments which, taken in conjunction with the accompanying drawings, illustrate by way of example the principles of the present invention.

### BRIEF DESCRIPTION OF THE DRAWINGS

FIG. 1 is an elevated view, partially in section, of a prior art stent mounted on a rapid-exchange delivery catheter and positioned within an artery;

FIG. 2 is an elevated view, partially in section, similar to that shown in FIG. 1 wherein the present invention is expanded within an artery so that the stent embeds within the artery wall;

FIG. 3 is an elevated view, partially in section, showing the expanded stent after withdrawal of the rapid-exchange delivery catheter;

FIG. 4 is a plan view of a flattened stent of one embodiment of the invention which illustrates the pattern of rings and links;

FIG. 5 is an elevated perspective view of the stent pattern of FIG. 4 is a cylindrical configuration corresponding to an actual stent;

FIG. 6 is a graph of the stiffness of a stent against the strut length;

FIG. 7 is an expanded view of a portion of the plan view of FIG. 4 showing the various parameters that affect the stiffness of the stent; and

FIG. 8 is a second embodiment of the invention illustrating a double mirror configuration.

### DETAILED DESCRIPTION OF THE PREFERRED EMBODIMENTS

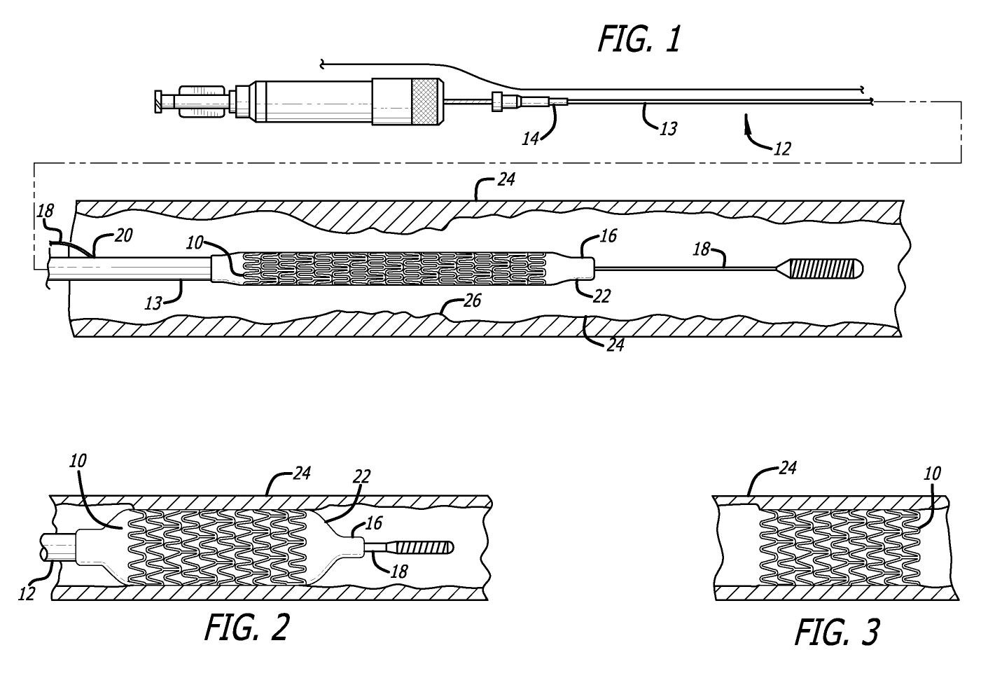

FIG. 1 depicts one environment of the present invention showing stent 10 mounted on a catheter assembly 12 which is used to deliver the stent and implant it in a body lumen, such as a coronary artery, peripheral artery, or other vessel or lumen within the body. The catheter assembly includes a catheter shaft 13 which has a proximal end 14 and a distal end 16. The catheter assembly is configured to advance through the patient's vascular system by advancing over a guide wire by any of the well known methods of an over the wire system (not shown) or a well known rapid exchange catheter system, such as the one shown in FIG. 1.

Catheter assembly 12 as depicted in FIG. 1 is of the well known rapid exchange type which includes an RX port 20 where the guide wire 18 will exit the catheter. The distal end of the guide wire 18 exits the catheter distal end 16 so that the catheter advances along the guide wire on a section of the catheter between the RX port 20 and the catheter distal end 16. As is known in the art, the guide wire lumen that receives the guide wire is sized for receiving various diameter guide wires to suit a particular application. The stent 10 is mounted on an expandable member 22 such as an inflatable balloon and is crimped tightly thereon so that the stent and expandable member 22 present a low profile diameter for delivery through go the arteries.

As shown in FIG. 1, a partial cross-section of an artery 24 is shown with a small amount of plaque 26 that has been previously treated by an angioplasty or other repair procedure. Stent 10 of the present invention may be used to repair a diseased or damaged arterial wall that has plaque 26 as shown in FIG. 1, or a dissection, or a flap as is commonly found in the coronary arteries, peripheral arteries, and other vessels.

In a typical procedure to implant stent 10, the guide wire 18 is advanced through the patient's vascular system by well known methods so that the distal end of the guide wire is advanced past the plaque 26 or diseased area. Prior to implanting the stent 10, the cardiologist may wish to perform an angioplasty procedure or other procedure (i.e., atherectomy) in order to open the vessel and remodel the diseased area. Thereafter, the stent delivery catheter assembly 12 is advanced over the guide wire 18 so that the stent is positioned in the target area. The expandable member 22 is inflated by well known means so that it expands radially outwardly and in turn expands the stent 10 radially outwardly until the stent 10 bears against the vessel wall. The expandable member 22 is then deflated and the catheter withdrawn from the patient's vascular system, leaving the stent in place to dilate the body lumen. The guide wire 18 typically is left in the lumen for post-dilation procedures, if any, and subsequently is withdrawn from the patient's vascular system. As depicted in FIGS. 2 and 3, the inflatable member 22 is fully inflated with the stent 10 expanded and pressed against the vessel wall, and in FIG. 3, the implanted stent remains in the vessel after the balloon has been deflated and the catheter assembly and guide wire have been withdrawn from the patient.

The stent 10 serves to hold open the artery after the catheter is withdrawn, as illustrated by FIG. 3. Due to the formation of the stent from an elongated tubular member in this particular embodiment, the undulating components of the stent are relatively thin in transverse cross-section, so that when the stent is expanded, it is pressed into the wall of the artery and as a result does not interfere significantly with the blood flow through the artery. The stent is pressed into the wall of the artery and will eventually be covered with endothelial cell growth which further minimizes blood flow interference. The undulating portion of the stent provides good tacking characteristics to prevent stent movement within the artery. Furthermore, the closely spaced cylindrical rings at regular intervals provide uniform support for the wall of the artery, and consequently are well adapted to tack up and hold in place small flaps or dissections in the wall of the artery, as illustrated in FIGS. 2 and 3.

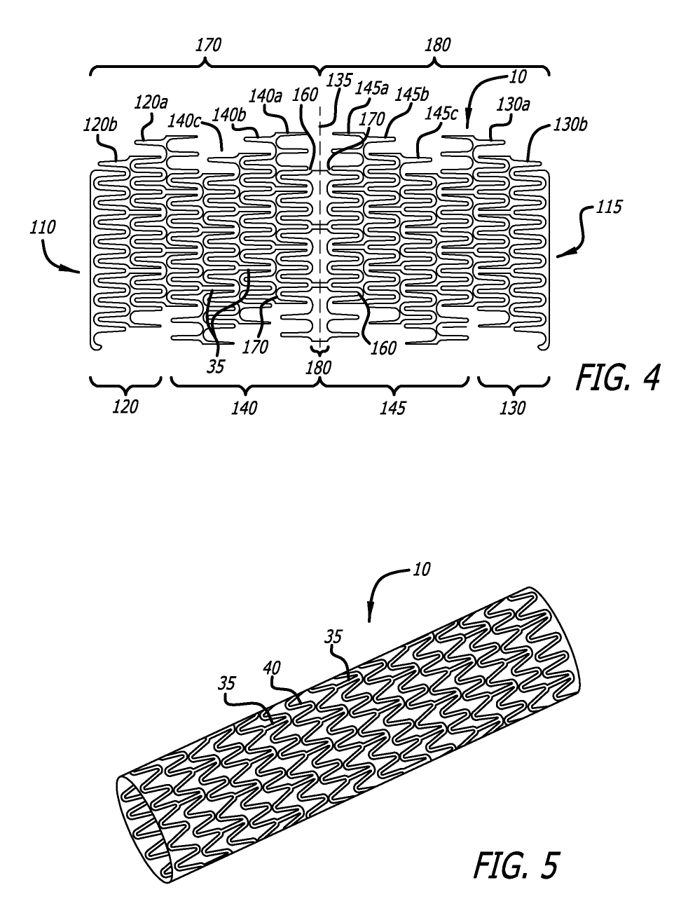

In keeping with the present invention, FIGS. 4 and 5 depict stent 10 having a particular undulation pattern and configuration. The stent embodiments and patterns as disclosed herein are illustrative and by way of example only. Other patterns embodying the inventions discussed herein can vary and still incorporate the stent retention and flexibility features of the present invention. Referring to FIG. 4, stent 10 is shown in a flattened condition so that the pattern can be clearly viewed, even though the stent is in a cylindrical form in use, such as shown in FIG. 5. The stent is typically formed from a tubular member, however, it can be formed from a flat sheet such as shown in FIG. 4 and rolled into a cylindrical configuration as shown in FIG. 5.

In FIGS. 4 and 5, stent 10 is shown in plan form and cylindrical form so that the pattern can be clearly viewed. The stent 10 is made up of a plurality of cylindrical body rings 40 which extend circumferentially around the stent when it is in a tubular form. Each cylindrical body ring 40 has a cylindrical ring proximal end and a cylindrical ring distal end. Typically, since the stent is laser cut from a tube there are no discreet parts such as the described cylindrical rings and links. However, it is beneficial for identification and reference to various parts to refer to the cylindrical rings and links and other parts of the stent as follows.

Each cylindrical body ring 40 defines a cylindrical plane which is a plane defined by the proximal and distal ends of the ring and the circumferential extent as the cylindrical ring travels around the cylinder. Each cylindrical ring 40 includes a cylindrical outer wall surface which defines the outermost surface of the stent, and a cylindrical inner wall surface which defines the innermost surface of the stent. The cylindrical plane follows the cylindrical outer wall surface. Adjacent rings are interconnected by a longitudinal links or struts 35 that are substantially straight and substantially aligned with the longitudinal axis of the stent. The longitudinal struts 35 are circumferentially spaced around the stent 10, such as for example in 30, 60, 90, or 120 degree intervals.

The stent 10 of the present invention has at its proximal end 110 one or more end rings 120, and at its distal end 115 one or more end rings 130. At the midpoint of the stent is a plane of symmetry 135 dividing the stent into a proximal region 170 and a distal region 180. Between the proximal end rings 120 and the plane of symmetry 135 are a plurality of intermediate rings 140. Similarly, between the distal end rings 130 and the plane of symmetry 135 are a plurality of intermediate rings 145. The number of end rings 120 (the example of FIG. 4 shows two) equals the number of end rings 130, and the number of intermediate rings 140 (the example of FIG. 4 shows four) equals the number of intermediate rings 145. Moreover, the ring 140a adjacent the plane of symmetry on the proximal side is a mirror reflection of the ring 145a adjacent the plane of symmetry on the distal side. The peaks 160 on the ring 140a correspond to the valleys 170 on the ring 145a and the valleys 170 on the ring 140a correspond to the peaks 160 on the ring 145a. Similarly, the rings once removed from the immediately adjacent rings to the plane of symmetry are mirror images where the peaks and valleys of ring 140b match up and correspond with the valleys and peaks of ring 145b, and so forth. The end rings 120a and 120b are also mirror reflections of end rings 130a and 130b, respectively. As such, the plane of symmetry divides the stent in the axial direction into two equal halves that are mirror images of each other. Each ring on the stent has a twin that is equal distant from the plane of symmetry 135 and are mirror images of each other.

A gap 180 between the peak 160 of the rings 140a and the valleys 170 of the rings 145a represents a greater spacing between adjacent rings at the plane of symmetry 135 than between any other two rings. This gap 180 creates a slightly enlarged window where additional balloon material can penetrate through the stent as the stent is crimped onto the balloon for anchor the stent on the balloon during the delivery process. The gap 180 locates the additional balloon material forced into the crimped stent at a first preferred location at the center of the stent, where torque loading due to lateral forces that may dislodge the stent is minimized.

It is desirable to design the stent 10 to have a higher stiffness at the proximal and distal ends, and a lower stiffness in the intermediate sections. A higher stiffness on the ends will promote a more uniform or homogeneous expansion of the balloon as it expands the stent, since a uniformly stiff stent tends to expand quicker at the ends and slower in the middle. This can cause difficulties in properly locating the stent as it may shift or slide if the contact with the lumen wall is not uniform. Moreover, uneven expansion can lead to damage of the lumen wall as the stent will have larger diameters in some locations while portions of the stent will be under deployed.

The formula for the calculation of the radial force F is given by the equation:

F=f*k

where f is the deflection of the strut and k is the constant of stiffness. One can calculate the constant of stiffness from the following:

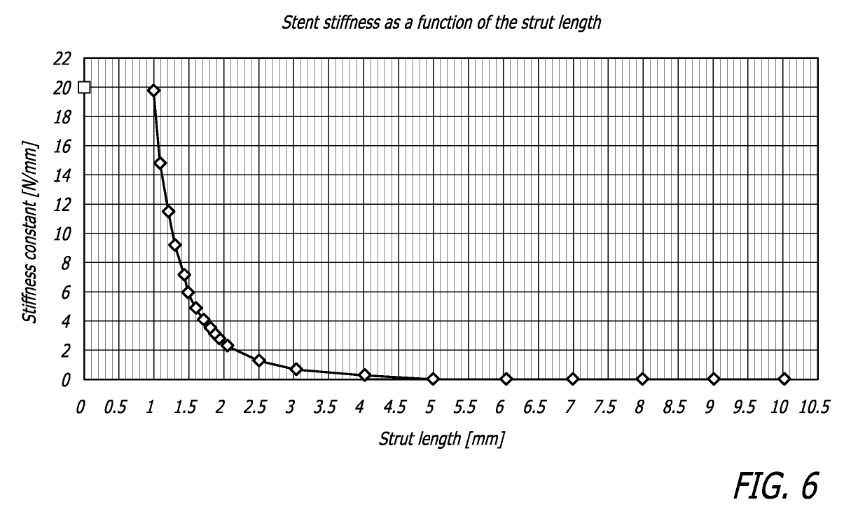

k=(E*s³*t)/(n*l³)

where E is Young's modulus, s is the strut width, t is the wall thickness, n is the amount of rings and l is the strut length. It is much more difficult to design a stent that has different proximal, middle, and distal strut widths or wall thicknesses. It is much easier to change the strut length to achieve different stiffnesses along the stent. FIG. 6 illustrates a graph of the stent stiffness (“k”) as a function of strut length (“l”), and it can be readily seen that a small change in the strut length can impact the stent's radial stiffness by a large amount percentage wise. For example, a strut length of 1.5 mm corresponds with a stiffness of 6 N/mm, whereas a strut length of 1.7 mm results in a stiffness of 4 N/mm, or a fifty percent decrease in stiffness for a 0.2 mm change in the strut length. A stent with a stiffness of 6 N/mm has beneficial flexibility characteristics and a high radial force in the strut's intermediate sections, whereas a stent with a stiffness of 4 N/mm has greater push stability. The present invention provides the benefits of both stiffnesses in a single stent.

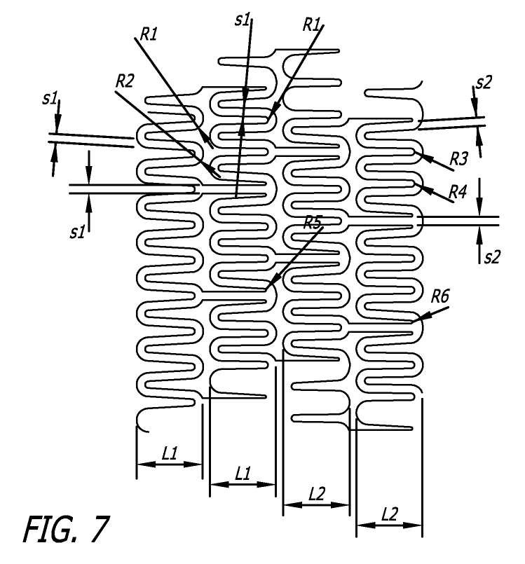

FIG. 7 illustrates a first preferred embodiment of the end rings 120 having a stiffness k1 and the intermediate rings 140 having a stiffness k2. The distance along the longitudinal axis of the stent between the peak 160 and the valley 170 of the end rings 120 is designated L1, and the distance between the peak and valley of the intermediate rings 140 is designated L2. In the prior art stents, L1 was equal to L2. In the present invention, L1 is less than L2 resulting in a stiffness of the end rings 120 being higher than the stiffness of the intermediate rings 140. In this manner, the end rings 120 have an associated stiffness k1 that is greater than the stiffness k2 that is associated with the intermediate rings 140, and the stiffness k1 of the end rings 130 is greater than the associated stiffness k2 of the intermediate rings 145. The stent 10 thus has the properties wherein the ends of the stent have greater stiffness coefficients and are stiffer while the intermediate portion of the stent have a lower stiffness coefficient and is less stiff, leading to a more uniform expansion while improving both flexibility and pushability.

Although adjusting the strut length is a preferred mode of varying the stiffness, other stent geometry can be employed to vary the stiffness of the stent at the ends in comparison with the interior section. Still referring to FIG. 7, the thickness of the ring's sinusoidal member as well as the respective curvatures of the sinusoidal members can also be adjusted to create the variable stiffnesses of the present invention. It is understood that the various parameters discussed below can be changed individually or in combination in arriving at the stiffnesses, and that it is not necessary to alter all of the parameters to achieve the variable stiffnesses.

The strut thickness is designated “S”, and the prior art stents had a constant strut thickness throughout the stent. However, to create a variable stiffness stent, the thickness of the strut S1 of the end rings 120 is greater than the thickness S2 of the intermediate rings 140, resulting in a higher stiffness for the end rings 120 as compared with the intermediate rings 140. The radius of curvature of the outer surface of the peaks and valleys, designated R1 on the end rings 120 and R3 on the intermediate rings 140 were constant and equal. To create a higher stiffness, the radius of curvature R1 on the end rings 140 is greater than the radius of curvature R3 on the intermediate rings 140. Additionally, the radius of curvature R2 on the inner surface of the peaks and valleys of the end rings 120 is increased to be greater than the radius of curvature R4 on the inner surface of the intermediate rings 140. The radius of curvature R5 of the inner surface of a peak that envelopes a strut Thus, one or more of the following equations are contemplated by the present invention:

L1<L2

S1>S2

R1>R3

R2>R4

R5<R6

Using one or more of the characteristics above as illustrated in FIG. 7, a stiffness associated with the end rings 120, 130 of the stent are greater than a stiffness associated with the intermediate rings 140, 145 of the stent.

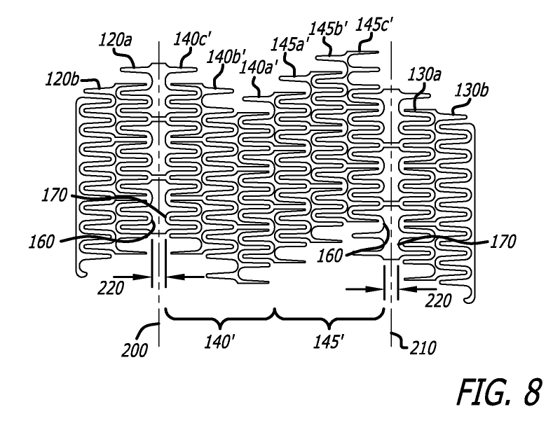

FIG. 8 illustrates another embodiment of the invention having a double mirror configuration. Here, end rings 120a,b and 130a,b are similar to the stent of FIG. 4. In the double mirror configuration of FIG. 8, a plane of symmetry borders each transition from end ring to intermediate ring. For example, a first plane of symmetry 200 is located between end ring 120a and intermediate ring 140c′. The peaks 160 of the end ring 120a match up with and correspond with the valleys 170 of the intermediate ring 140c′, creating a mirror image. Similarly, a second plane of symmetry 210 is located between end ring 130a and intermediate ring 145c′, creating a mirror image between intermediate ring 145c′ and end ring 130a. End ring 130b is a mirror image of intermediate ring 145b′. As in the single mirror design, the double mirror design has a first stiffness associated with the end rings 120, 130, and a lower stiffness associated with the intermediate rings 140′,145′. A gap or spacing 220 about each plane of symmetry provides for increased balloon material to extend into and possibly through the stent, helping to secure the stent to the balloon as discussed above in connection with gap 180. Alternatively, as with the initial embodiment, the radial stiffness of the end portions could be less than the radial stiffness of the intermediate rings if the application favored such a configuration.

The stent 10 of the present invention can be mounted on a balloon catheter similar to the catheter shown in the prior art device in FIG. 1. The stent is tightly compressed or crimped onto the balloon portion of the catheter and remains tightly crimped onto the balloon during delivery through the patient's vascular system. When the balloon is expanded, the stent expands radially outwardly into contact with the body lumen, for example, a renal or coronary artery. When the balloon portion of the catheter is deflated, the catheter system is withdrawn from the patient and the stent remains implanted in the artery. Similarly, if the stent of the present invention is made from a self-expanding metal alloy, such as nickel-titanium or the like, the stent may be compressed or crimped onto a catheter and a sheath (not shown) is placed over the stent to hold it in place until the stent is ready to be implanted in the patient. Such sheaths are well known in the art. Further, such a self-expanding stent may be compressed or crimped to a delivery diameter and placed within a catheter. Once the stent has been positioned within the artery, it is pushed out of the catheter or the catheter is withdrawn proximally and the stent held in place until it exits the catheter and self-expands into contact with the wall of the artery. Balloon catheters and catheters for delivering self-expanding stents are well known in the art.

The stent 10 of the present invention can be made in many ways. One method of making the stent is to cut a thin-walled tubular member, such as stainless steel tubing to remove portions of the tubing in the desired pattern for the stent, leaving relatively untouched the portions of the metallic tubing which are to form the stent. The stent also can be made from other metal alloys such as tantalum, nickel-titanium, cobalt-chromium, titanium, shape memory and superelastic alloys, and the Nobel metals such as gold or platinum. In accordance with the invention, it is preferred to cut the tubing in the desired pattern by means of a machine-controlled laser as is well known in the art.

The stent of the present invention also can be made from metal alloys other than stainless steel, such as shape memory alloys. Shape memory alloys are well known and include, but are not limited to, nickel-titanium and nickel-titanium-vanadium. Any of the shape memory alloys can be formed into a tube and laser cut in order to form the pattern of the stent of the present invention. As is well known, the shape memory alloys of the stent of the present invention can include the type having superelastic or thermoelastic martensitic transformation, or display stress-induced martensite. These types of alloys are well known in the art and need not be further described here. Importantly, a stent formed of shape memory alloys, whether the thermoelastic or the stress-induced martensite-type, can be delivered using a balloon catheter of the type described herein, or be delivered via a catheter without a balloon or a sheath catheter.

The present invention stent is ideally suited, for example, for drug delivery (i.e., delivery of a therapeutic agent) since it has a uniform surface area which ensures uniform distribution of drugs. Typically, a polymer is coated onto the stent of the type disclosed in U.S. Pat. Nos. 6,824,559 and 6,783,793 which are incorporated herein by reference. These bioactive agents can be any agent, which is a therapeutic, prophylactic, or diagnostic agent. These agents can have anti-proliferative or anti-inflammatory properties or can have other properties such as antineoplastic, antiplatelet, anti-coagulant, anti-fibrin, antithrombonic, antimitotic, antibiotic, antiallergic, antioxidant as well as cytostatic agents. Representative embodiments of the active component include actinomycin D (available from Sigma-Aldrich; or Cosmegen® available from Merck) or derivatives, analogs or synonyms thereof, such as dactinomycin, actinomycin IV, actinomycin I.sub.1, actinomycin X.sub.1, and actinomycin C.sub.1; podophyllotoxins such as etoposide and teniposide (Bristol Myers Squibb and Sigma Chemical); cephalotin (Bristol Myers Squibb); trapidil; ticlopidine (Danbury Pharma, Genpharm); tranilast (SmithKline Beecham and LG Chemical Kissei, Japan); IIb-IIIa inhibitors such as eptifibatide (COR therapeutic); clobetasol (Glaxo Wellcome); COX-2 inhibitors such as celecoxib (CELEBREX) (Searle and Pfizer) and rofecoxib (VIOXX) (Merck); PGE1 or alprostadil (Bedford); bleomycin; ENDOSTATIN (EntreMed); ANGIOSTATIN (EntreMed); thalidomide; 2-methoxyestraidol (EntreMed and Sigma Chemical) curcimin (the major constituent of turmeric power extract from the rhizomes of the plant Curcuma longa L found in south and southeast tropical Asia); cisplatin (Sigma Chemical); dipyridamole; tirofiban; verapamil; vitronectine; argatroban; and carboplatin (Sigma Chemical). Additionally corticosteroids such as anti-inflammatory glucocorticoids including clobetasol, diflucortolone, flucinolone, halcinonide, and halobetasol can also be used.

In one embodiment, faster acting non-steroidal anti-inflammatory agents such as naproxen, aspirin, ibuprofen, fenoprofin, indomethacin, and phenylbutazone can be used in conjunction with the glucocorticoids. The use of a non-steroidal anti-inflammatory agent is useful during the early stages of the inflammation in response to a mechanically mediated vascular injury. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include methyl rapamycin, ABT-578, 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack, N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax a (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacore® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl(4-amino-TEMPO), estradiol, anticancer agents, dietary supplements such as various vitamins, and a combination thereof. Examples of anti-inflammatory agents including steroidal and non-steroidal anti-inflammatory agents include tacrolimus, dexamethasone, clobetasol, combinations thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium.

Other therapeutic substances or agents which may be appropriate include alpha-interferon, bioactive RGD, and genetically engineered epithelial cells. The foregoing substances can also be used in the form of prodrugs or co-drugs thereof. The bioactive agents also include metabolites of the foregoing substances and prodrugs of these metabolites. The foregoing substances are listed by way of example and are not meant to be limiting. Other active agents which are currently available or that may be developed in the future are equally applicable.

While particular forms of the invention have been illustrated and described, it will be apparent to those skilled in the art that various modifications can be made without departing from the spirit and scope of the invention. Accordingly, it is not intended that the invention be limited except by the appended claims

I claim:

1. A stent, comprising:
undulating rings aligned about a common axis and links extending between adjacent rings, wherein said undulating rings and links are arranged so as to define a plane of symmetry extending through the center of the stent, and perpendicular to said axis.

2. A stent, comprising:
undulating rings aligned about a common axis and links extending between adjacent rings, wherein the rings that define a distal end and a proximal end of said stent are mirror images of one another.

3. The stent of claim 2 wherein the rings that define a distal end and a proximal end have a radial stiffness that is greater than a radial stiffness of the rings that are intermediate thereof.

4. The stent of claim 3 wherein a strut length of the rings defining a proximal end and distal end is greater than a strut length of rings that are intermediate thereof.

5. The stent of claim 3 wherein a thickness of the rings defining a proximal end and distal end is greater than a thickness of rings that are intermediate thereof.

6. The stent of claim 3 wherein a radius of curvature of an outer surface of the rings defining a proximal end and distal end is greater than a radius of curvature of an outer surface of rings that are intermediate thereof.

7. The stent of claim 3 wherein a radius of curvature of an inner surface of the rings defining a proximal end and distal end is greater than a radius of curvature of an inner surface of rings that are intermediate thereof.

8. The stent of claim 3 wherein a radius of curvature of an outer surface of the rings defining a proximal end and distal end is greater than a radius of curvature of an outer surface of rings that are intermediate thereof.

9. The stent of claim 3 wherein a radius of curvature of between a strut and a peak or valley of the rings defining a proximal end and distal end is less than a radius of curvature between a strut and a peak or valley of the rings that are intermediate thereof.

10. The stent of claim 2 having a plane of symmetry at a midpoint thereof, and having a larger gap between adjacent rings at the plane of symmetry than with other adjacent rings.

11. A stent having a plurality of interconnected sinusoidal rings connected by longitudinal struts to form a cylindrical structure comprising:
a plane of symmetry at a midpoint of the stent; and
at least one ring forming a proximal end portion and at least one ring forming a distal end portion, the proximal end portion and the distal end portion having a common radial stiffness that is greater than a radial stiffness of rings intermediate of the proximal and distal end portions.

12. The stent of claim 11 further comprising a gap between rings bordering the plane of symmetry that is greater than a gap between any other two adjacent rings.

13. The stent of claim 11 wherein the radial stiffness of rings intermediate of the proximal and distal end portions are is constant for each ring.

14. The stent of claim 11 wherein a length of a strut interconnecting the at least one ring of the proximal end portion is less than a length of a strut interconnecting rings intermediate of the proximal end portion and the plane of symmetry.

15. The stent of claim 15 wherein the length of a strut interconnecting the at least one ring of the proximal end portion is equal to a length of a strut interconnecting the at least one ring of the distal end portion.

16. A stent having a plurality of interconnected sinusoidal rings connected by longitudinal struts to form a cylindrical structure comprising:
at least one ring forming a proximal portion and at least one ring forming a distal portion, the proximal portion and the distal portion having a radial stiffness that is greater than a radial stiffness of rings intermediate of the proximal and distal portions; and
a first plant of symmetry immediately adjacent a distal end of the proximal portion and a second plane of symmetry immediately adjacent the proximal end of the distal portion.

17. The stent of claim 16 wherein the radial stiffness of the proximal portion is equal to the radial stiffness of the distal portion.

18. The stent of claim 17 wherein a radial stiffness of the rings intermediate of the proximal and distal portions is constant.
